(19) United States
(12) Patent Application Publication
(10) Pub. No.: US 2014/0296943 A1
(43) Pub. Date: Oct. 2, 2014

(54) METHOD FOR CONTROLLING BLOOD GLUCOSE LEVELS AND DIGESTION CYCLES

(71) Applicant: Biological Illumination, LLC, Satellite Beach, FL (US)

(72) Inventors: Fredric S. Maxik, Indialantic, FL (US); David E. Bartine, Cocoa, FL (US); Robert R. Soler, Cocoa Beach, FL (US)

(73) Assignee: Biological Illumination, LLC, Satellite Beach, FL (US)

(21) Appl. No.: 14/208,370

(22) Filed: Mar. 13, 2014

Related U.S. Application Data
(60) Provisional application No. 61/785,209, filed on Mar. 14, 2013.

(51) Int. Cl. A61N 5/06

(52) U.S. Cl. CPC A61N 5/0613; A61N 2005/0626
USPC 607/88

(57) ABSTRACT

A method of affecting a physiological rhythm is provided. The method may include the steps of receiving an indication of a physiological condition of a patient, determining if the physiological condition is outside an expected status, receiving an indication of a physiological rhythm status of the patient, determining a physiological rhythm phase of the patient responsive to the indicated physiological rhythm status, determining a treatment light configured to affect at least one of an advance and delay physiological rhythm response in the patient, and causing the treatment light to be emitted onto the patient.

### RELATED APPLICATIONS

This application is related to and claims the benefit under 35 U.S.C. §119(e) of U.S. Provisional Patent Application Ser. No. 61/785,209 titled Method for Controlling Blood Glucose Production filed Mar. 14, 2013 (Attorney Docket No.(588.00031), the content of which is incorporated by reference herein in its entirety.

### FIELD OF THE INVENTION

The present invention relates to systems and methods for controlling blood glucose production using a lighting device.

### BACKGROUND

Increasingly, the extent to which physiological rhythms govern the functioning of various anatomical systems is better understood. Moreover, research suggests these physiological rhythms may be manipulated by providing external stimuli, providing cues to the body of an observer of the light that may result in a response in the physiological rhythm. There is a need in the art for systems and methods for identifying when a physiological rhythm response is desirable, and to affect such a response through the provision of external stimuli.

### SUMMARY OF THE INVENTION

With the above in mind, embodiments of the present invention may advantageously provide a method for affecting a physiological rhythm. More specifically, the method according to embodiments of the present invention may advantageously allow for physiological rhythms to be manipulated by providing an external stimulus.

The method may include receiving an indication of a physiological condition of a patient, determining if the physiological condition is outside an expected status, receiving an indication of a physiological rhythm status of the patient, determining a physiological rhythm phase of the patient responsive to the indicated physiological rhythm status, determining a treatment light configured to affect at least one of an advance and delay physiological rhythm response in the patient and causing the treatment light to be emitted onto the patient.

The step of receiving the indication of a physiological condition of a patient may comprise receiving a measurement of a physiological substance of the patient. The physiological substance may be selected from the group consisting of blood glucose, fasting blood glucose, and insulin.

The physiological rhythm status may be an indicator selected from the group consisting of body temperature, activity level, chryptochrome level, leptin level, melatonin level, blood glucose level, insulin level, and cortisol level. The physiological rhythm may be a circadian rhythm. Furthermore, the physiological rhythm response may comprise affecting a change in at least one of IGF-1 secretion rate, insulin breakdown rate, gluconeogenesis rate, glycogenolysis rate, and glycogenesis rate in the patient.

The step of determining if the physiological condition is outside an expected status may comprise determining a time of day associated with the indication of a physiological condition, determining an expected status associated with the determined time of day, and comparing the expected status to the indication of a physiological condition. The step of receiving an indication of a physiological rhythm status of the patient may comprise receiving a signal from a device worn by the patient.

Additionally, embodiments of the present invention are directed to a method of affecting a blood glucose level in a patient comprising the steps of receiving a blood glucose level of a patient, determining if the blood glucose level is outside a target range, receiving an indication of a physiological rhythm status of the patient, determining a physiological rhythm phase of the patient responsive to the indicated physiological rhythm status, determining a treatment light configured to affect at least one of an advance and delay physiological rhythm response in the patient to thereby affect the blood glucose level of the patient, and causing the treatment light to be emitted onto the patient. The step of determining if the blood glucose level is outside a target range may comprise determining a time of day associated with the received blood glucose level of the patient, determining an expected blood glucose level associated with the determined time of day, and comparing the expected blood glucose level to the received blood glucose level of the patient.

When the blood glucose level is determined to be above the target range, and when the physiological rhythm phase is determined to be one of a day phase, an evening phase, or a night phase, the treatment light may be configured to affect a delay response. When the blood glucose level is determined to be above the target range, and when the physiological rhythm phase is determined to be a morning phase, the treatment light may be configured to affect an advance response. When the blood glucose level is determined to be below the target range, and when the physiological rhythm phase is determined to be one of a day phase, an evening phase, or a night phase, the treatment light may be configured to affect an advance response. When the blood glucose level is determined to be below the target range, and when the physiological rhythm phase is determined to be a morning phase, the treatment light may be configured to affect a delay response.

Additionally, embodiments of the present invention are directed to a method of affecting a blood glucose level in a patient by controlling the emission of light onto the patient via use of a computerized device operatively coupled to a lighting device that may be configured to emit the light. The method may comprise the steps of receiving a first signal indicating a blood glucose level of a patient via a communication device associated with the computerized device, determining a time of day associated with the received blood glucose level of the patient, determining an expected blood glucose level associated with the determined time of day, and comparing the expected blood glucose level to the received blood glucose level of the patient. The method may further comprise the steps of receiving a second signal comprising an indication of a physiological rhythm status of the patient from a device worn by the patient, the indication of physiological rhythm status being at least one of body temperature and activity level via the communication device, determining a physiological rhythm phase of the patient responsive to the indicated physiological rhythm status, determining a treatment light configured to affect at least one of an advance and delay physiological rhythm response in the patient to thereby affect the blood glucose level of the patient and transmitting a third signal to the lighting device causing the treatment light to be emitted thereby.

### BRIEF DESCRIPTION OF THE DRAWINGS

FIG. 1 illustrates melatonin and cortisol levels across a 24-hour period depicting circadian rhythms for melatonin and cortisol.

FIG. 2 illustrates the light spectra of conventional light sources in comparison to a predicted melatonin suppression action spectrum for polychromatic light.

FIG. 3 illustrates various types of circadian responses in levels of melatonin.

FIG. 4 illustrates circadian rhythms for blood glucose and insulin levels across a 24-hour period.

FIG. 5 illustrates normal, delay, and advance insulin circadian rhythms across a 24-hour period.

FIG. 6 illustrates normal, delay, and advance blood glucose circadian rhythms across a 24-hour period.

FIG. 7 illustrates a flowchart according to a method of the invention.

FIG. 8 illustrates a flowchart according to an alternative method of the invention.

### DETAILED DESCRIPTION OF THE PREFERRED EMBODIMENT

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Those of ordinary skill in the art realize that the following descriptions of the embodiments of the present invention are illustrative and are not intended to be limiting in any way. Other embodiments of the present invention will readily suggest themselves to such skilled persons having the benefit of this disclosure. Like numbers refer to like elements throughout.

Although the following detailed description contains many specifics for the purposes of illustration, anyone of ordinary skill in the art will appreciate that many variations and alterations to the following details are within the scope of the invention. Accordingly, the following embodiments of the invention are set forth without any loss of generality to, and without imposing limitations upon, the claimed invention.

In this detailed description of the present invention, a person skilled in the art should note that directional terms, such as “above,” “below,” “upper,” “lower,” and other like terms are used for the convenience of the reader in reference to the drawings. Also, a person skilled in the art should notice this description may contain other terminology to convey position, orientation, and direction without departing from the principles of the present invention.

An embodiment of the invention, as shown and described by the various figures and accompanying text, provides a method for treating a condition that is responsive to changes in circadian rhythm. More specifically, an embodiment of the invention provides a method for treating a condition that is responsive to changes in circadian rhythm caused by a circadian response to light.

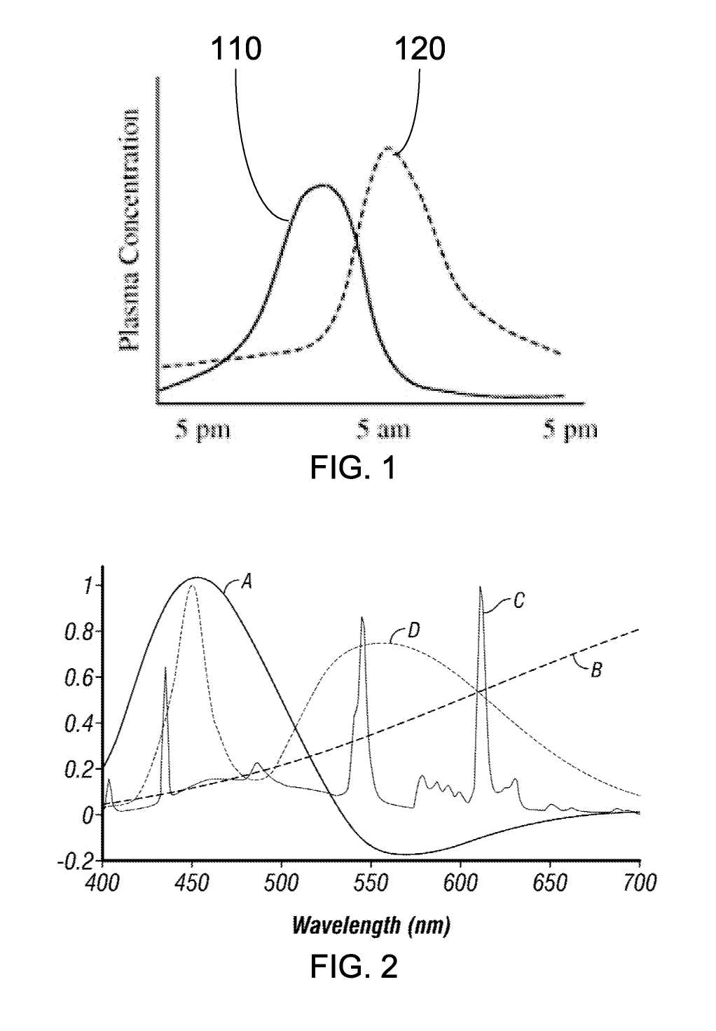

Referring now to FIG. 1, an illustration of blood plasma concentration levels of various substances having a circadian rhythm across a 24-hour period is presented. More specifically, plot 110 illustrates the concentration of melatonin, and plot 120 illustrates the concentration of cortisol. Both melatonin and cortisol have been found to have a circadian rhythm, repeating across an approximately 24-hour period. See, for instance, Melatonin as a Chronobiotic, by Josephine Arendt and Debra Jean Skene, Sleep Medicine Reviews, Vol. 9, Iss. 1, February 2005, pages 25-39, the content of which is incorporated herein by reference in its entirety, and which may be found at http://www.sciencedirect.com/science/article/pii/S1087079204000474. Also, see, for instance, Review: Replication of cortisol circadian rhythm: new advances in hydrocortisone replacement therapy, Sharon Chan and Miguel Debono, Therapeutic Advances in Endocrinology and Metabolism, August 2010, Vol. 1 No. 3, pages 129-138, the content of which is incorporated by reference in its entirety, and which may be found at http://tae.sagepub.com/content/1/3/129.abstract. Moreover, it is known in the art that both melatonin and cortisol are indicative of the circadian rhythm of the human body. As such, by determining the level of at least one of melatonin or cortisol, an associated phase of the circadian rhythm may similarly be determined. Furthermore, it is appreciated that there are other bioindicators of circadian rhythm that may be measured to determine the phase of the circadian rhythm. Examples of other bioindicators include, without limitation, cryptochrome, leptin, blood glucose, insulin, and core body temperature. Moreover, physiological rhythms and their current phase other than circadian rhythm may be indicated by any of the aforementioned bioindicators, as well as any others known in the art. Furthermore, it is understood in that art that changes in circadian rhythm may have correlative changes in the blood concentration of other substances. Such substances may include, by example only and not by means of limitation, glucose, insulin, IGF-1. See, for example, BMAL1 and CLOCK, Two Essential Components of the Circadian Clock, Are Involved in Glucose Homeostasis, R. Daniel Rudic, Peter McNamara, Anne-Maria Curtis, Raymond C Boston, Satchidananda Panda, John B Hogenesch, and Garret A FitzGerald, PLoS Biol 2(11): e377. Doi: 10.1371/journal.pbio.0020377, which may be found at http://www.plosbiology.org/article/info %3Adoi %2F10.1371%2Fjournal.pbio.00203 77.

Additionally, in some embodiments, the determination of the current phase of a circadian rhythm may be made by means or methods other than measurement of a physiological substance. For example, wearable devices including sensors capable of monitoring physical statuses of the wearer, including movement, activity level, temperature, and skin salinity are known in the art, and the wearable devices are further capable of generating a signal indicating the measurement each of these statuses. Furthermore, it is known in the art that certain measurements of the aforementioned statuses may be indicative of the wearer being in a particular phase of a physiological rhythm, such as a circadian rhythm. Accordingly, the physiological rhythm status of a patient may be determined by receiving an indication of said status from a device worn by the patient. More information regarding wearable devices may be found in U.S. Provisional Patent Application Ser. No. 61/948,185 titled System for Dynamically Adjusting Circadian Rhythm Responsive to Scheduled Events and Associated Methods filed Mar. 5, 2014 (Attorney Docket No. 818.00001), the content of which is incorporated in its entirety herein by reference, except to the extent disclosure there is inconsistent with disclosure herein.

Additionally, it is known that melatonin may be suppressed by exposure to certain wavelengths of light. Referring now to FIG. 2, an illustration of light spectra and its effect on melatonin is presented. As shown by plot A, a predicted maximum suppression is experienced at wavelengths around about 460 nm. In other words, a light source having a spectral component between about 420 nm and about 480 nm is expected to cause melatonin suppression.

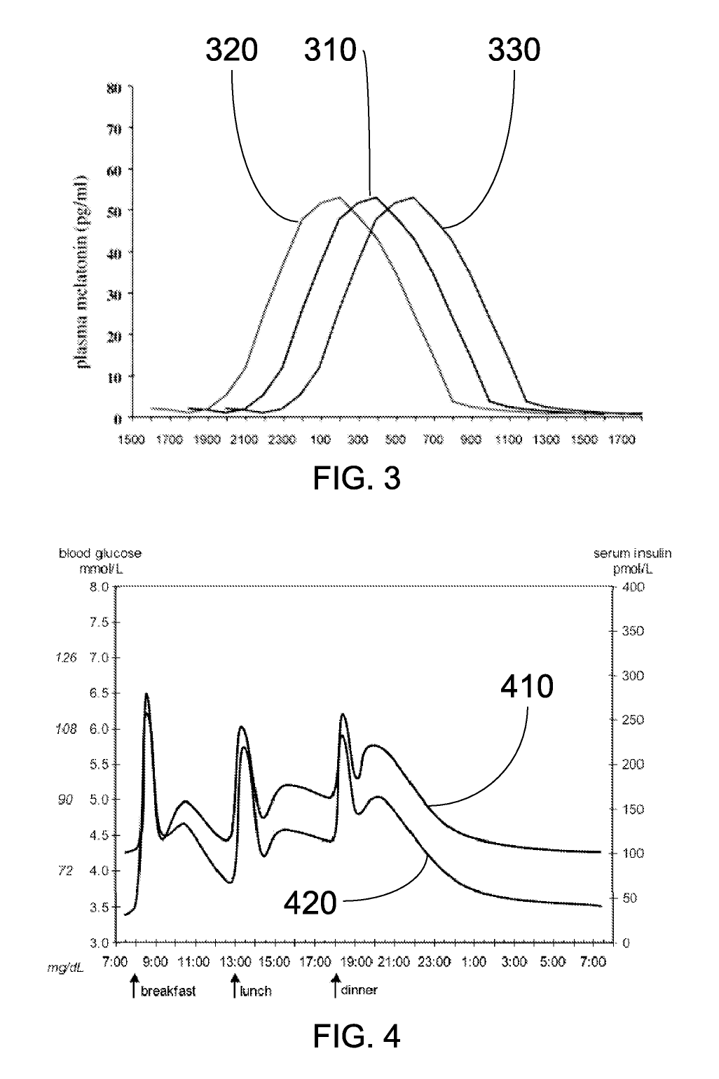

Referring now to FIG. 3, an illustration of circadian responses is presented. Plot 310 illustrates a standard plasma melatonin concentration across a 24-hour period representing melatonin levels associated with a normal circadian rhythm. It is appreciated that, while the circadian responses illustrated in FIG. 3 are related to the circadian rhythm associated with melatonin, similar responses in circadian rhythm for the bioindicators recited hereinabove may be accomplished. As illustrated by plot 310, there is a peak concentration of melatonin at about 3 A.M. of about approximately 50 pg/mL, and a nadir concentration at about 8 P.M. of about approximately less than 1 pg/mL. In plot 310, the concentration of melatonin begins to sharply increase at about 9 P.M.

Plot 320 illustrates a plasma melatonin concentration across a 24-hour period after an advance response. As can be seen in plot 320, the peak melatonin concentration occurs at about 1 A.M., and the nadir occurs at about 6 P.M. As such, the physiological rhythm response illustrated by plot 320 represents an advance of approximately 2 hours with respect to a normal circadian rhythm as represented by plot 310. An advance physiological rhythm response may be accomplished by a variety of methods, including providing illumination that is configured to reduce melatonin suppression. An example of a device capable of providing such illumination is presented in U.S. patent application Ser. No. 13/652,207 titled LED Lamp for Producing Biologically-Corrected Light, the content of which is incorporated by reference herein in its entirety. The illumination may be provided as a light treatment, wherein the recipient, such as a patient, is illuminated with a high concentration of light configured to cause the advance physiological response. In some embodiments, the light treatment may be for a duration within the range from about 1 minute to about 60 minutes. Furthermore, the emitted light may be perceived and/or incident upon the retina of the patient, and/or the emitted light may be incident upon the skin of the patient.

Plot 330 illustrates a plasma melatonin concentration across a 24-hour period after a delay physiological rhythm response. As can be seen in plot 330, the peak melatonin concentration occurs at approximately 6 A.M., and the nadir occurs at approximately 10 P.M. As such, the physiological rhythm response illustrated by plot 330 represents a delay of approximately 2 hours with respect to a normal circadian rhythm as represented by plot 310. A delay physiological rhythm response by a variety of methods, including providing illumination this is configured to cause melatonin suppression. It is understood in the art the providing illumination having a spectral component within the range of wavelengths from about 420 nm to about 480 nm may cause melatonin suppression, resulting in a delay circadian response.

Referring now to FIG. 4, an illustration of levels of glucose and insulin in blood across a 24-hour period is presented. Plot 410 illustrates blood glucose levels, and plot 420 illustrates blood insulin levels. It can be seen that the insulin levels 420 closely track the glucose levels 410. Moreover, each of the glucose and insulin levels 410, 420 are correlated with the consumption of meals, with significant increases in both at the meal times indicated. As the meals may be generally considered to occur at approximately the same time daily, a circadian rhythm may be determined from both the glucose and the insulin plots 410, 420.

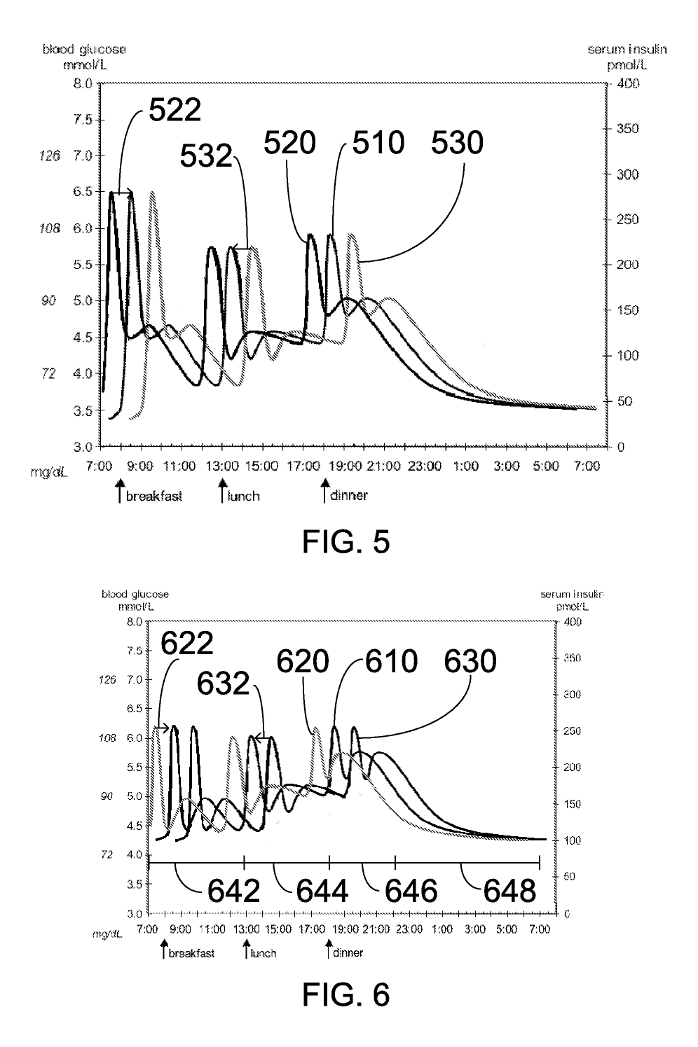

Referring now to FIG. 5, an illustration of a circadian response in the insulin circadian rhythm is presented. In FIG. 5, plot 510 illustrates a normal insulin circadian rhythm, as presented in plot 420 of FIG. 4. Plot 520 illustrates an insulin circadian rhythm that is in advance of the normal insulin circadian rhythm 510. Accordingly, it may be desired to affect a delay response, represented by reference 522. The delay response 522 may be accomplished by any method, including, but not limited to, affecting a delay response in another physiological rhythm. It is contemplated and included within the scope of the invention that the delay circadian response 522 may be accomplished by affecting a delay response in any other physiological rhythm, including, but not limited to, a melatonin circadian rhythm, a cryptochrome circadian rhythm, a leptin circadian rhythm, a cortisol circadian rhythm, and a core body temperature circadian rhythm.

Additionally, it is appreciated that there may be a latency period between affecting the delay response in the other physiological rhythm and the insulin circadian rhythm. Accordingly, it is contemplated and included within the scope of the invention for a latency period to transpire before the delay response 522 in the insulin circadian rhythm 520 is affected.

Similar to the advance circadian rhythm 520, plot 530 illustrates an insulin circadian rhythm that is in delay of the normal insulin circadian rhythm 510. Accordingly, it may be desired to affect and advance response 532. Similar to the delay response 522, the advance response 532 may be accomplished by affecting a delay response in another physiological rhythm, including those rhythms referenced hereinabove.

Referring now to FIG. 6, an illustration of a circadian response in the glucose circadian rhythm is presented. Similar to the circadian responses illustrated in FIG. 5, plot 610 illustrates a normal glucose circadian rhythm across a 24-hour period, plot 620 illustrates a glucose circadian rhythm in advance of the normal glucose circadian rhythm 610, and plot 630 illustrates a glucose circadian rhythm in delay of the normal glucose circadian rhythm 610. Similar to the circadian responses illustrated in FIG. 5, the advance glucose circadian rhythm 620 may be shifted to align with the normal glucose circadian rhythm 610 by affecting a delay response 622, and the delay glucose circadian rhythm 630 may be shifted to align with the normal glucose circadian rhythm 610 by affecting an advance response 632.

Each of the delay response 622 and the advance response 632 may be accomplished by affecting a delay or advance response in another physiological rhythm. In addition to the physiological rhythms identified above, the delay and advance responses 622, 632 may be accomplished by affecting a respective delay or advance response in a physiological rhythm associated with at least one of the pancreas and the liver. More specifically, a physiological response in at least one of the pancreas and the liver may be accomplished through a light treatment, as described hereinabove, of a patient for which the delay or advance response 622, 632 is desired. In some embodiments, the physiological rhythm response may cause the pancreas to alter the secretion of insulin. In some other embodiments, the physiological rhythm response may cause the liver to alter at least one of an IGF-1 secretion rate, an insulin breakdown rate, a gluconeogenesis rate, a glycogenolysis rate, and a glycogenesis rate.

Shifting of the blood glucose circadian rhythm may be desirable as a method for treating a condition related to blood glucose levels. An example of such a condition is diabetes, more particularly diabetes mellitus type 2. This condition is exemplary only, and any condition that may be treated by a shift in a physiological rhythm is contemplated and included within the scope of the invention.

The determination as to whether an advance or delay physiological response is needed may be based upon the level of a physiological substance, being defined as a physiological rhythm marker level, such as a circadian rhythm marker level. Types of physiological substances that may be measured may include, but is not limited to, cryptochrome, leptin, melatonin, blood glucose, insulin, and cortisol. Additionally, a physiological condition may similarly serve as a circadian rhythm marker level indicate the need for an advance or delay response. For example, a core body temperature may be measured, and a delay or advance response may be indicated. For either physiological substances or conditions, a variance outside a target range may indicate the need for an advance or delay response. As is shown in FIGS. 3-6, the level of the physiological substance may vary with time. Accordingly, the target range may vary with the time of day at which the physiological substance level is determined. For example, a blood glucose level may be determined at a time after waking but prior to the consumption of breakfast, known as a fasting blood glucose level. In some embodiments, the target range for a fasting blood glucose level may be from about 82 mg/dL to about 110 mg/dL.

Referring now back to FIG. 6, the normal glucose circadian rhythm may be divided into a plurality of phases. In some embodiments, the blood glucose circadian rhythm may include a morning phase 642 being defined generally as about from a time prior to consuming a morning meal to about a time prior to the consumption of a mid-day meal. The blood glucose circadian rhythm may additionally include a day phase 644 being generally as about from consumption of a mid-day meal to about prior to consumption of an evening meal. Furthermore, the blood glucose circadian rhythm may additionally include an evening phase 646 being generally defines as from about consumption of an evening meal to about prior to falling asleep in the evening. Finally, the blood glucose circadian rhythm may include a night phase 648 being generally defined as from approximately falling asleep to about a time prior to consuming a morning meal.

As described hereinabove, a determination of the blood glucose level may indicate being within a phase of the blood glucose circadian rhythm. Additionally, the determination of the phase of the blood glucose circadian rhythm may be made by a comparison of the blood glucose level along with the time of day during which the measurement is taken. Furthermore, the advance and delay responses 622, 632 may result in a respective advance or delay of the current phase of the blood glucose circadian rhythm. More specifically, when an advance response is affected, a patient to whom the advance response is affected may, for example, shift from a morning phase to a day phase, from a day phase to an evening phase, etc. Similarly, when a delay response is affected, a patient to whom the advance response is affected may, for example, shift from a day phase to a morning phase, from an evening phase to a day phase, etc.

Where the blood glucose level is above the target range for a particular phase of the blood glucose circadian rhythm, either a delay or advance response may be indicated. Where the blood glucose circadian rhythm is one of the day phase, the evening phase, or the night phase, a delay response may be indicated. Where the blood glucose circadian rhythm is in the morning phase, an advance response may be indicated.

Similarly, where the blood glucose level is above the target range for a particular phase of the blood glucose circadian rhythm, either a delay or advance response may be indicated. Where the blood glucose circadian rhythm is in one of the day phase, the evening phase, or the night phase, an advance response may be indicated. Where the blood glucose circadian rhythm is in the morning phase, a delay response may be indicated.

Furthermore, in some embodiments, the delay and advance circadian responses may be associated with a change in insulin production, as disclosed hereinabove. Generally, insulin production is increased during the day phase, reduced during the morning phase and the evening phase, and reduced further during the night phase. Where a blood glucose level is determined to be above a target range for a particular phase of the blood glucose circadian rhythm, a circadian response shifting toward the day phase may be indicated. Where a blood glucose level is determined to be below a target range for the particular phase of the blood glucose circadian rhythm, a circadian response shifting toward the night phase may be indicated.

It is appreciated that all of the aforementioned physiological rhythm responses, such as advance and delay responses, may be accomplished through the administration of a light treatment as described hereinabove. Accordingly, where an advance or delay response is indicated, a light treatment affecting such a response may be understood to be desirable and/or administered to a patient indicating a need for such a response.

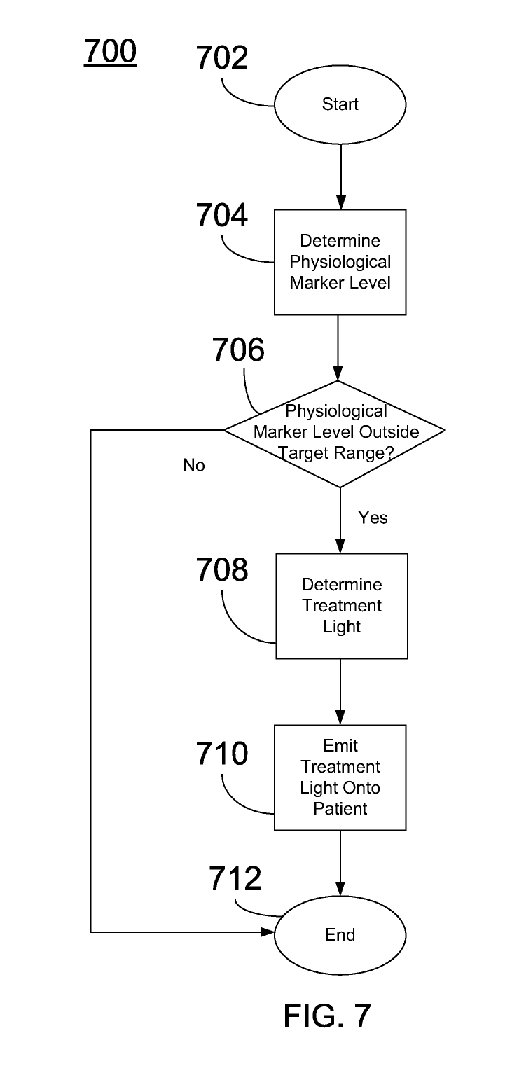

Referring now to FIG. 7, in accordance with an embodiment of the invention, a method is illustrated by flowchart 700. Starting at Block 702, the method may proceed to Block 704, where a physiological marker level of a patient may be determined. This may be accomplished in any number of ways depending on the physiological marker that is to be determined. The most common way, however, to determine such a physiological marker is to draw the blood of the patient and conduct an analysis. Those skilled in the art, however, will appreciate that this can be determined in any number of ways, and the present invention is not intended to be limited to physiological markers that are determined by blood analysis. At Block 706, it may be determined whether the physiological marker level is outside a target range. If, at Block 706, it is determined the physiological marker level is not outside the target range, the method may end at Block 712. If, at Block 706, it is determined that the physiological marker level is outside the target range, a treatment light having a spectral power distribution configured to generate a response in a physiological rhythm may be determined at Block 708. The method may proceed to Block 710, where a light having the spectral power distribution of the treatment light determined at Block 708 may be emitted so as to be incident upon the patient. The method may then end at Block 712.

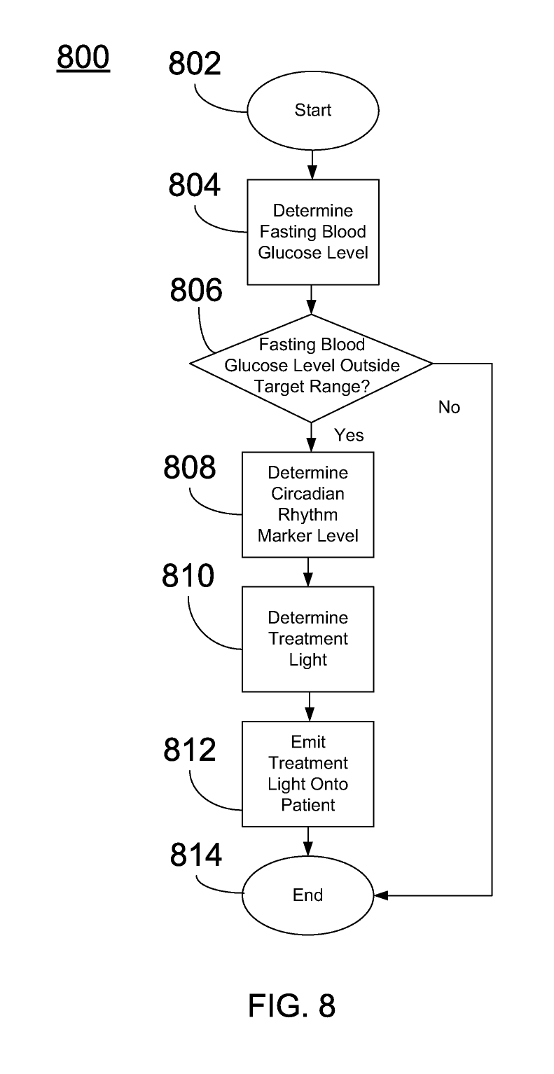

Referring now to FIG. 8, a method of treating a blood glucose-related condition is illustrated by flowchart 800. Starting at Block 802 the method may proceed to Block 804 where a fasting blood glucose level of a patient may be determined. The method may proceed to Block 806 where it may be determined whether the fasting blood glucose level is outside a target range. If, at Block 806, it is determined the fasting blood level is not outside the target range, the method may end at Block 814. If, at Block 806, it is determined the fasting blood glucose level is outside the target range, the method may then proceed to Block 808 where a circadian rhythm marker of the patient may be determined. The method may then proceed to Block 810 where a treatment light having a spectral power distribution configured to generate a desired circadian response is determined. Proceeding to Block 812, a light having the spectral power distribution of the treatment light determined at Block 810 may be emitted so as to be incident upon the patient. The method may then end at Block 814.

Throughout the above disclosure, it is referenced that various measurements may be made, determinations of physiological conditions and rhythm statuses may be made, and a treatment light may be determined and emitted by a lighting device so as to affect a physiological rhythm response in a patient. Accordingly, it is contemplated and included within the scope of the invention that a system comprising a computerized device in communication with one or more lighting devices may be configured to receive the various inputs, including a signal comprising an indication of a physiological condition of a patient, such as blood glucose level, as well as any other physiological substance disclosed hereinabove. Additionally, the computerized device may further be configured to determine if the physiological condition of the patient is outside an expected status. The computerized device may also be configured to determine a time of day associated with the received physiological condition to facilitate determination of if the physiological condition is outside an expected status by comparison thereof. Furthermore, the computerized device may be configured to receive a signal comprising an indication of a physiological rhythm status of the patient. In some embodiments, the computerized device may include a communication device positioned in communication with a device worn by the patient, as described hereinabove, and may receive said physiological rhythm status to determine a physiological rhythm phase of the patient. The computerized device may further be configured to determine a treatment light configured to affect at least one of an advance or delay response in the patient to thereby affect a physiological response in the patient. The computerized device may further be configured to transmit a signal to the lighting device causing the treatment light to be emitted thereby, Additional information regarding such computerized devices and lighting devices may be found in U.S. Provisional Patent Application Ser. No. 61/948,185 and U.S. patent application Ser. No. 13/652,207, each of which is incorporated by reference hereinabove.

Additionally, some embodiments of the invention may include computer software that may be stored on a computer-readable medium that may perform the above-described methods by receiving the various inputs, establishing communication with a lighting device using computer hardware that is capable of reading and executing the software, and causing the treatment light as described hereinabove to be emitted.

Some of the illustrative aspects of the present invention may be advantageous in solving the problems herein described and other problems not discussed which are discoverable by a skilled artisan.

While the above description contains much specificity, these should not be construed as limitations on the scope of any embodiment, but as exemplifications of the presented embodiments thereof. Many other ramifications and variations are possible within the teachings of the various embodiments. While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best or only mode contemplated for carrying out this invention, but that the invention will include all embodiments falling within the scope of the appended claims. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited. Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced item.

Thus the scope of the invention should be determined by the appended claims and their legal equivalents, and not by the examples given.

What is claimed is:

1. A method of affecting a physiological rhythm comprising the steps of:
receiving an indication of a physiological condition of a patient;
determining if the physiological condition is outside an expected status;
receiving an indication of a physiological rhythm status of the patient;
determining a physiological rhythm phase of the patient responsive to the indicated physiological rhythm status;
determining a treatment light configured to affect at least one of an advance and delay physiological rhythm response in the patient; and
causing the treatment light to be emitted onto the patient.

2. The method according to claim 1 wherein the step of receiving the indication of a physiological condition of a patient comprises receiving a measurement of a physiological substance of the patient.

3. The method according to claim 2 wherein the physiological substance is selected from the group consisting of blood glucose, fasting blood glucose, and insulin.

4. The method according to claim 1 wherein the physiological rhythm status is an indicator selected from the group consisting of body temperature, activity level, chryptochrome level, leptin level, melatonin level, blood glucose level, insulin level, and cortisol level.

5. The method according to claim 1 wherein the physiological rhythm is a circadian rhythm.

6. The method according to claim 1 wherein the physiological rhythm response comprises affecting a change in at least one of IGF-1 secretion rate, insulin breakdown rate, gluconeogenesis rate, glycogenolysis rate, and glycogenesis rate in the patient.

7. The method according to claim 1 wherein the step of determining if the physiological condition is outside an expected status comprises:
determining a time of day associated with the indication of a physiological condition;
determining an expected status associated with the determined time of day; and
comparing the expected status to the indication of a physiological condition.

8. The method according to claim 1 wherein the step of receiving an indication of a physiological rhythm status of the patient comprises receiving a signal from a device worn by the patient.

9. A method of affecting a blood glucose level in a patient comprising the steps of:
receiving a blood glucose level of a patient;
determining if the blood glucose level is outside a target range;
receiving an indication of a physiological rhythm status of the patient;
determining a physiological rhythm phase of the patient responsive to the indicated physiological rhythm status;
determining a treatment light configured to affect at least one of an advance and delay physiological rhythm response in the patient to thereby affect the blood glucose level of the patient; and
causing the treatment light to be emitted onto the patient.

10. The method according to claim 9 wherein the physiological rhythm status is an indicator selected from the group consisting of body temperature, activity level, chryptochrome level, leptin level, melatonin level, blood glucose level, insulin level, and cortisol level.

11. The method according to claim 9 wherein the physiological rhythm is a circadian rhythm.

12. The method according to claim 9 wherein the physiological rhythm response comprises at least one of affecting a change in IGF-1 secretion rate, insulin breakdown rate, gluconeogenesis rate, glycogenolysis rate, and glycogenesis rate in the patient.

13. The method according to claim 9 wherein the step of determining if the blood glucose level is outside a target range comprises:
determining a time of day associated with the received blood glucose level of the patient;
determining an expected blood glucose level associated with the determined time of day; and
comparing the expected blood glucose level to the received blood glucose level of the patient.

14. The method according to claim 9 wherein the step of receiving an indication of the physiological rhythm status of the patient comprises receiving a signal from a device worn by the patient.

15. The method according to claim 9 wherein, when the blood glucose level is determined to be above the target range, and when the physiological rhythm phase is determined to be one of a day phase, an evening phase, or a night phase, the treatment light is configured to affect a delay response.

16. The method according to claim 9 wherein, when the blood glucose level is determined to be above the target range, and when the physiological rhythm phase is determined to be a morning phase, the treatment light is configured to affect an advance response.

17. The method according to claim 9 wherein, when the blood glucose level is determined to be below the target range, and when the physiological rhythm phase is determined to be one of a day phase, an evening phase, or a night phase, the treatment light is configured to affect an advance response.

18. The method according to claim 9 wherein, when the blood glucose level is determined to be below the target range, and when the physiological rhythm phase is determined to be a morning phase, the treatment light is configured to affect a delay response.

19. A method of affecting a blood glucose level in a patient by controlling the emission of light onto the patient via use of a computerized device operatively coupled to a lighting device that is configured to emit the light, the method comprising the steps of:
receiving a first signal indicating a blood glucose level of a patient via a communication device associated with the computerized device;
determining a time of day associated with the received blood glucose level of the patient;
determining an expected blood glucose level associated with the determined time of day;
comparing the expected blood glucose level to the received blood glucose level of the patient;
receiving a second signal comprising an indication of a physiological rhythm status of the patient from a device worn by the patient, the indication of physiological rhythm status being at least one of body temperature and activity level via the communication device;
determining a physiological rhythm phase of the patient responsive to the indicated physiological rhythm status;
determining a treatment light configured to affect at least one of an advance and delay physiological rhythm response in the patient to thereby affect the blood glucose level of the patient; and
transmitting a third signal to the lighting device causing the treatment light to be emitted thereby.

20. The method according to claim 19 wherein the physiological rhythm response comprises affecting a change in at least one of IGF-1 secretion rate, insulin breakdown rate, gluconeogenesis rate, glycogenolysis rate, and glycogenesis rate in the patient.
